SEQUENCE LISTING (6 sequences)

SEQ ID NO: 1 - DNA, 24 bp, Artificial Sequence
  sense primer for albumin gene
gagaaggtcaccaagtgctgtagt

SEQ ID NO: 2 - DNA, 24 bp, Artificial Sequence
  antisense primer for albumin gene
ctgggagtgtgcagatatcagagt

SEQ ID NO: 3 - DNA, 21 bp, Artificial Sequence
  sense primer for CK19 gene
accatgcagaacctgaacgat

SEQ ID NO: 4 - DNA, 20 bp, Artificial Sequence
  antisense primer for CK19 gene
cacctccagctcgccattag

SEQ ID NO: 5 - DNA, 21 bp, Artificial Sequence
  sense primer for beta-actin gene
gaagatttggcaccacacttt

SEQ ID NO: 6 - DNA, 20 bp, Artificial Sequence
  antisense primer for beta-actin gene
ttgaatgtagtttcatggat

(19) United States
(12) Patent Application Publication
(10) Pub. No.: US 2006/0223181 A1
(43) Pub. Date: Oct. 5, 2006

(54) Method for separating hepatic progenitor cell

(76) Inventors: Eiji Miyoshi, Toyonaka-shi (JP); Katsuhisa Noda, Toyonaka-shi (JP); Naoyuki Taniguchi, Toyonaka-shi (JP); Norio Hayashi, Kawanishi-shi (JP)

Correspondence Address: BIRCH STEWART KOLASCH & BIRCH, PO BOX 747, FALLS CHURCH, VA, 22040-0747, US

(21) Appl. No.: 11/450,319

(22) Filed: Jun. 12, 2006

Related U.S. Application Data
(62) Division of application No. 10/822,760, filed on Apr. 13, 2004.

(30) Foreign Application Priority Data
May 21, 2003 (JP) 2003-142807

(51) Int. Cl. C12N 5/08

(52) U.S. Cl. 435/370

(57) ABSTRACT

The present invention relates to a method for identifying a pluripotent hepatic progenitor cell, a method for separating the pluripotent hepatic progenitor cell and a method for producing the pluripotent hepatic progenitor cell.

### CROSS-REFERENCE TO RELATED APPLICATIONS

The present application is a 37 C.F.R. §1.53(b) divisional of U.S. patent application Ser. No. 10/822,760 filed Apr. 13, 2004, which claims priority on Japanese Patent Application No. 2003-142807, filed May 21, 2003, the entire contents of which are hereby incorporated by reference and for which priority is claimed under 35 U.S.C. §120.

### BACKGROUND OF THE INVENTION

1. Field of the Invention

The present invention relates to a method for identifying a pluripotent hepatic progenitor cell, a method for separating the pluripotent hepatic progenitor cell and a method for producing the pluripotent hepatic progenitor cell.

2. Description of Related Art

The liver is known as an organ having very high regenerability. In addition, by recent progression in cellular biology, it has been revealed that homeostasis of the liver is controlled based on a stem cell system. A technique for identifying or separating a hepatic stem cell is an important technology to make hepatic regenerative medicine feasible.

Recently, a method for separating a stem cell has been developed with advancement of cell separation techniques such as flow cytometry. In particular, regarding a hematopoietic stem cell, a separation method utilizing various antigens on the cell surface has been developed. In addition, examples of reconstruction of bone marrow from one separated cell have been also reported.

An oval cell, a hepatoblast, a marrow-derived cell and the like have been considered as a candidate for a stem cell in the liver. All of these cells can differentiate into a hepatocyte, and are considered to be involved in pathology of hepatopathy. The above-mentioned method using flow cytometry has been introduced in the separation of a hepatic stem cell in the liver (or a stem cell-like cell), whereby a method for selectively separating and collecting a stem cell (stem cell-like cell) has been invented, wherein the stem cell exists in trace amounts in the liver and cannot be morphologically discriminated. For example, Taniguchi et al. have reported that c-Met⁺CD49f^{+/low}c-Kit⁻CD45⁻Ter119⁻cells in a non-hemocyte fraction (CD45⁻Ter119⁻cells) in fetal mouse liver are separated by flow cytometry using a fluorescence-labeled antibody specific for each antigen, and the resulting cell fraction contains pluripotent cells having high proliferation potency and capable of differentiating into two different cells, hepatocytes and bile ductal cells [see, for example, The Journal of Cell Biology, vol. 156, pp. 173-184 (2002)]. Alternatively, Ferris [see, for example, JP-A No. 2002-520015 (WO 00/03001) has reported a method for separating a hepatic stem cell from hepatic parenchymal cells, comprising a step of selecting a cell expressing an antigen selected from the group consisting of laminin, desmoprachin I, intercellular adhesion molecule (CCAM), carcinoembryonic antigen (CEA), dipeptidyl peptidase-4, γglutamyl transpeptidase (gGT), VLA-2, VLA-3, VLA-5 and VLA-6.

However, it is current circumstance that a stem cell in the liver is not clearly defined and that the above-mentioned candidate cells for a hepatic stem cell are merely found. In addition, it is current circumstance that what cell surface antigen is expressed at each stage of differentiation from a stem cell to a mature cell has not been sufficiently analyzed.

On the other hand, sugar chains are distributed on the cell surface in the form of a glycoprotein or a glycolipid, and play an important role in an interaction between cells or between a cell and an extracellular matrix, intracellular signaling, and targeting of a protein. Recently, many glycosyltransferases involved in the biosynthesis of sugar chains have been cloned, whereby the information on biological function of sugar chains has been accumulated.

### BRIEF SUMMARY OF THE INVENTION

In one aspect, the present invention relates to providing a method for identifying a pluripotent hepatic progenitor cell, comprising detecting a sugar chain expressed on the pluripotent hepatic progenitor cell. According to the identification method of the present invention, there can be achieved, at least one of: the identification of, for example, a cell constituting the liver, specifically, for example, a cell at least having an ability of differentiating into a hepatocyte, a hepatic non-parenchymal cell and the like, namely, a pluripotent hepatic progenitor cell in a cell population containing various cells; the effective separation of the hepatic progenitor cell from a cell population containing various cells; improvement in purity of the pluripotent hepatic progenitor cell, and the like.

In another aspect, the present invention relates to providing a method for separating a pluripotent hepatic progenitor cell, comprising sorting a pluripotent hepatic progenitor cell using as an index a sugar chain expressed on a pluripotent hepatic progenitor cell. According to the separation method of the present invention, there can be achieved at least one of: the separation of a pluripotent hepatic progenitor cell from a cell population containing various cells; the collection of a pluripotent hepatic progenitor cell in high purity; the collection of a pluripotent hepatic progenitor cell with stable quality, and the like.

In still another aspect, the present invention relates to providing a method for producing a composition comprising a pluripotent hepatic progenitor cell, comprising the step of separating a pluripotent hepatic progenitor cell by the above-mentioned identification method. According to the production method of the present invention, there can be achieved at least one of: the collection of a composition comprising a pluripotent hepatic progenitor cell, which is suitable in application to any one of: the tissue regeneration by cell transplantation, ex vivo introduction of a nucleic acid molecule, a gene, for example a therapeutic gene, or a derivative thereof; the production of a transplantation tissue; the application use for research etc., the collection of a composition comprising a pluripotent hepatic progenitor cell at high purity and the like.

The present invention relates to:

[1] a method for identifying a pluripotent hepatic progenitor cell, comprising detecting a sugar chain expressed on the pluripotent hepatic progenitor cell;

[2] the method according to the above item [1], wherein the sugar chain is detected by using a protein capable of binding to the sugar chain expressed on the pluripotent hepatic progenitor cell;

[3] the method according to the above item [2], wherein the protein is a lectin capable of binding to the sugar chain expressed on the pluripotent hepatic progenitor cell;

[4] the method according to the above item [1], wherein the sugar chain expressed on the pluripotent hepatic progenitor cell comprises a sugar chain structure recognized by at least one lectin selected from the group consisting of kidney bean lectin, wheat germ lectin, lentil lectin and Aleuria aurantia lectin;

[5] the method according to the above item [1], wherein the sugar chain is detected by using an antibody capable of binding to the sugar chain expressed on the pluripotent hepatic progenitor cell;

[6] the method according to the above item [1], wherein the sugar chain is detected via an expression of an enzyme involved in the synthesis of the sugar chain expressed on the pluripotent hepatic progenitor cell;

[7] the method according to the above item [6], wherein the expression of the enzyme is detected by at least one means selected from the group consisting of the measurement of an enzyme activity, the measurement of the amount of the enzyme protein and the measurement of an amount of mRNA from a gene encoding the enzyme;

[8] the method according to the above item [6], wherein the enzyme is N-acetylglucosaminyltransferase III, sialyltransferase or a-1,6 fucosyltransferase;

[9] a method for separating a pluripotent hepatic progenitor cell, comprising sorting the pluripotent hepatic progenitor cell using as an index a sugar chain expressed on the pluripotent hepatic progenitor cell;

[10] the method according to the above item [9], wherein the pluripotent hepatic progenitor cell is sorted by using a protein capable of binding to the sugar chain expressed on the pluripotent hepatic progenitor cell;

[11] the method according to the above item [10], wherein the protein is a lectin capable of binding to the sugar chain expressed on the pluripotent hepatic progenitor cell;

[12] the method according to the above item [10], wherein the sugar chain expressed on the pluripotent hepatic progenitor cell comprises a sugar chain structure recognized by at least one lectin selected from the group consisting of kidney bean lectin, wheat germ lectin, lentil lectin and Aleuria aurantia lectin;

[13] the method according to the above item [10], wherein the pluripotent hepatic progenitor cell is sorted by using an antibody capable of binding to the sugar chain expressed on the pluripotent hepatic progenitor cell; and

[14] a method for producing a composition comprising a pluripotent hepatic progenitor cell, comprising the step of separating the pluripotent hepatic progenitor cell by the method of any one of the above items [9to [13].

### BRIEF DESCRIPTION OF THE DRAWINGS

FIG. 1 is the result of FACS using FITC-labeled E4PHA, in the present invention, (a) RLE cell, (b) a hepatic cancer cell AH66, (c) a hepatocyte immediately after perfusion, (d) a hepatocyte non-parenchymal cell after culturing for 48 hours, and (e) a hepatic non-parenchymal cell after culturing for 48 hours.

FIG. 2 is the result of FACS using FITC-labeled LCA, in the present invention, (a) RLE cell, (b) a hepatocyte immediately after perfusion, (c) a hepatocyte non-parenchymal cell after culturing for 48 hours, and (d) a hepatic non-parenchymal cell after culturing for 48 hours.

### DETAILED DESCRIPTION OF THE INVENTION

According to the present invention, there can be provided tools for analyzing a change in a structure of a cell surface sugar chain, glycosyltransferase activity or gene expression in a process of differentiation from an undifferentiated cell of a hepatocyte to a mature cell, identifying a cell surface sugar chain marker specific for the pluripotent hepatic progenitor cell, and giving a hepatic stem cell (or a hepatic stem cell-like cell), namely, a hepatic progenitor cell by this sugar chain marker.

The present invention is based on the surprising finding found by the present inventors that a pluripotent hepatic progenitor cell, specifically, a glycoprotein on the cell surface has specifically high reactivity with kidney bean lectin (E4PHA), wheat germ lectin (WGA), lentil lectin (LCA) or Aleuria aurantia lectin (AAL), and is significantly different from a glycoprotein from a primary culture hepatocyte or a hepatic cancer cell. Further, the present invention is based on the surprising finding by the present inventors that a pluripotent hepatic progenitor cell can be identified by using the above-mentioned glycoprotein, specifically, a cell surface sugar chain antigen as a marker, and that the cell can be sorted using cell separation technique such as flow cytometry.

One aspect of the present invention is a method for identifying a pluripotent hepatic progenitor cell, comprising detecting a sugar chain expressed on the pluripotent hepatic progenitor cell.

Another aspect of the present invention is a method for separating a pluripotent hepatic progenitor cell, comprising sorting the pluripotent hepatic progenitor cell using as an index a sugar chain expressed on the pluripotent hepatic progenitor cell.

According to the identification method of the present invention, since a sugar chain expressed on a pluripotent hepatic progenitor cell is detected, there is exhibited an excellent effect that a pluripotent hepatic progenitor cell can be more efficiently identified from a cell population containing various cells at high specificity. Further, according to the identification method of the present invention, since the above-mentioned sugar chain is used as an index, there is exhibited an excellent effect that a pluripotent hepatic progenitor cell can be identified by a factor capable of specifically binding to, or associating with, the sugar chain or an event involving the sugar chain. Therefore, according to the identification method of the present invention, there can be achieved the improvement in purity of a pluripotent hepatic progenitor cell, the evaluation of purity, and the like. In addition, the identification method of the present invention is also useful for evaluating quality of a cell for transplantation or gene therapy for the liver.

In addition, according to the separation method of the present invention, since a sugar chain expressed on a pluripotent hepatic progenitor cell is used as an index upon sorting the pluripotent hepatic progenitor cell, there is exhibited an excellent effect that the pluripotent hepatic progenitor cell can be more efficiently separated from a cell population containing various cells at higher specificity. Therefore, according to the separation method of the present invention, a pluripotent hepatic progenitor cell with stable quality can be obtained. Thus, when a hepatic progenitor cell separated by the separation method of the present invention is utilized in tissue regeneration by cell transplantation, ex vivo introduction of a nucleic acid molecule, a gene, for example, a therapeutic gene or a derivative thereof, the production of a transplantation tissue, there can be achieved the regeneration of a substantially homogeneous tissue, a substantially uniform therapeutic effect, and a supply of a transplantation tissue with more stable quality.

The “pluripotent hepatic progenitor cell (multipotent hepatocyte precursor cell)” in the present invention refers to a cell having both multipotency meaning that the cell can differentiate into plural kinds of mature cells each having different functions in the liver and self-renewality meaning that the cell can reproduce the same cell as self. The “pluripotent hepatic progenitor cell” also includes a cell further having property of long life or immortality.

The “pluripotent hepatic progenitor cell” includes, for example, a cell which is the most undifferentiated cell in a cell population constituting the liver and has a multipotency in the liver, and the like. A candidate for such cell includes, for instance, a precursor cell, called as an oval cell, capable of differentiating into a biliary epithelial cell and a hepatocyte. In addition, the concept of the “pluripotent hepatic progenitor cell” encompasses a more undifferentiated cell having multipotency which can exist in a region surrounding portal vein. Further, the concept of the “pluripotent hepatic progenitor cell” encompasses a stem cell which can exist in a bone marrow cell or a peripheral blood and can differentiate into a hepatocyte. The methods for identifying and separating a pluripotent hepatic progenitor cell of the present invention are suitably applied to these undifferentiated and pluripotent hepatic progenitor cell candidates.

A material for separating a pluripotent hepatic progenitor cell is any organ, tissue or the like, without any particular limitation, as long as the material is expected to contain the cell, and includes, for instance, an adult liver tissue, a fetal liver tissue, a marrow tissue, a blood cell, a marrow cell, a peripheral blood cell, and the like. Further, according to the method for identifying or separating a hepatic progenitor cell of the present invention, a more undifferentiated and pluripotent cell can be separated by applying the present methods to a cell population fractionated by an existing cell fractionation method, a cell population obtained by the conventional differentiation method, or the like.

A source for the “material for separating the pluripotent hepatic progenitor cell” can be appropriately selected depending on the application use, and includes a mammal (e.g. primate such as human, monkey, chimpanzee and the like, mouse, rat, guinea pig, hamster, rabbit, dog and the like) and the like.

In the identification method and separation method of a pluriptipotent hepatic progenitor cell of the present invention, as described above, a sugar chain expressed (or existing) on the pluripotent hepatic progenitor cell can be detected by a factor capable of specifically binding to, or associating with, the sugar chain expressed on the pluripotent hepatic progenitor cell or via event involving the sugar chain.

The factor capable of specifically binding to, or associating with the sugar chain includes a protein capable of binding to, or associating with the sugar chain, an antibody capable of binding to the sugar chain (antibody against the sugar chain) and the like.

In one embodiment of the identification method of the present invention, the sugar chain expressed on the pluripotent hepatic progenitor cell is detected by using a protein capable of binding to the sugar chain. Specifically, in this embodiment of the identification method, a process comprising:

(A) contacting the “material for separating the pluripotent hepatic progenitor cell” with a protein capable of binding to the sugar chain expressed on the pluripotent hepatic progenitor cell, and

(B) detecting a cell with the protein bound thereto, in the mixture obtained in the step (A);

is performed to identify the cell with the protein bound thereto as a pluripotent hepatic progenitor cell.

In addition, in one embodiment of the separation method of the present invention, a pluripotent hepatic progenitor cell is sorted by using the protein capable of binding to the sugar chain expressed on the pluripotent hepatic progenitor cell. Specifically, in this embodiment of the separation method, a process comprising:

(a) contacting the “material for separating the pluripotent hepatic progenitor cell” with a protein capable of binding to the sugar chain expressed on the pluripotent hepatic progenitor cell, and

(b) sorting to separate a cell with the protein bound thereto, in the mixture obtained in the step (a);

is performed to sort the cell with the protein bound thereto as a pluripotent hepatic progenitor cell.

As the protein capable of binding to, or associating with the sugar chain, for example, a lectin can be used. Lectin is defined as a sugar-binding protein or a glycoprotein other than an immunoreaction product, which can aggregate cells or a complex carbohydrate. The lectin includes vegetable lectin and animal lectin. The lectin used in the present invention is not particularly limited, as long as it reacts (including, e.g. “binds” or “associates”) with the sugar chain expressed (or existing) on the surface of the pluripotent hepatic progenitor cell. In the present invention, a lectin capable of reacting with a sugar chain specifically expressed on the cell is particularly preferably used. Various lectins of which recognition of sugar chain structures (oligosaccharide structures) were investigated in detail have been commercially available for research of an oligosaccharide structure, and these lectins can be suitably used in the present invention. The lectin includes, for example, L-fucose-binding lectin; D-galactose, N-acetyl-D-galactosamine-binding lectin; D-mannose-binding lectin; di-N-acetylchitobiose-binding lectin; sialic acid-binding lectin and the like. Sugar chain recognition specificity of these lectins is described in detail, for example, in Methods in Enzymology, Vol. 230, pp. 66-86 edited by William J. Lennarz, Gerald W. Hart published by Academic Press, Inc. in 2002, and New Biochemistry Experimental Course 3, Carbohydrate I, Glycoprotein (first volume), pp. 3-29.

Lectin which is found to be useful in the identification method and the separation method of the present invention includes, for example, kidney bean lectin, wheat germ lectin, lentil lectin, Aleuria aurantia lectin and the like. Kidney bean lectin has a popular name of E4PHA, and is known to recognize and bind to a complex-type sugar chain structure having a bisecting GlcNAc structure. Wheat germ lectin has a popular name of WGA, and recognizes and binds to a hybrid-type or complex-type sugar chain structure having a sialic acid and/or bisecting GlcNAc structure. Lentil lectin has a popular name of LCA, and possesses strong affinity with a glycopeptide or a glycoprotein having a duplex complex-type sugar chain of structure in which α-L-fucose residue is bound to N-acetylglucosamine residue positioned at a reducing end, or a glycopeptide or a glycoprotein having a triplex complex-type sugar chain of a branch at C-2,6 of α-D-mannose residue. Aleuria aurantia lectin has a popular name of AAL, is a lectin specific for fucose residue and capable of binding to α-1,2 or α-1,3-L-fucose residue, and has strong affinity, particularly, with a sugar chain having α-1,6-L-fucose residue.

In the present invention, lectin can be used as it is, or lectins labeled with various labeling substances (e.g. fluorescent substance such as FITC, enzyme such as peroxidase, biotin and the like) can be used. Alternatively, in the present invention, plural kinds of lectins can be used in combination.

In the identification method of the present invention, in order to identify a pluripotent hepatic progenitor cell using a lectin as an index, for example, methods such as a lectin blotting method, a lectin column method, a cell staining method with labeled lectin, flow cytometry using labeled lectin are used. Details of the these methods are described, for example, in Glycobiology Experimental Protocol, Cell Technology supplementary volume, Experimental Protocol Series published by Syujunsha in 1996; Current Electrophoresis Experimental Method edited by Japanese Electrophoresis Society published by Ishiyaku Publishers, Inc. in 1999, and the like.

Lectin blotting can be carried out, for example, as follows: Cells to be analyzed are washed once with PBS (phosphate-buffered saline), and dissolved in TNE buffer (composition: 10 mM Tris-HCl, pH 7.8 containing 1 wt % NP-40, 0.15 M NaCl, 1 mM EDTA and PROTEASE INHIBITOR COCKTAIL (manufactured by Sigma)), to obtain a cell lysate. The cell lysate corresponding to an amount of 3 to 25 μg of a protein is electrophoresed on a 8 to 12 wt % SDS-polyacrylamide gel, and thereafter the separated substance is electrically blotted onto a nitrocellulose membrane. After completion of blotting, the resulting membrane is incubated to block in 3 wt % BSA (bovine serum albumin) at room temperature overnight. The nitrocellulose membrane is incubated at room temperature for 30 minutes to 2 hours, or at 4° C. for a few hours to overnight, in a solution in which labeled lectin, for example, biotinylated lectin is dissolved in TBS (Tris-buffered saline) containing 0.05 wt % Tween 20 (trade name). The biotinylated lectin may be, but not particularly limited to, a biotinylated lectin prepared, for example, by labeling lectin with a labeling substance, for example, a biotin by the conventional method, or a biotinylated lectin commercially available from, for example, Seikagaku Corporation. The membrane after incubation with the labeled lectin, for example, biotinylated lectin is washed three times with TBS containing 0.05 wt % Tween 20 (trade name) for 10 minutes. Thereafter, the membrane after washing is immersed in a solution obtained by appropriately diluting a peroxidase-labeled avidin with TBS containing 0.05 wt % Tween 20 (trade name), and incubated at room temperature for 30 minutes to 2 hours. The resulting membrane is washed three times with TBS containing 0.05 wt % Tween 20 (trade name) for 10 minutes. Next, the lectin bound to a glycoprotein on the membrane is detected by the conventional method depending on the labeling substance used in labeling. For example, when the biotinylated lectin is used, the lectin bound to a glycoprotein on the membrane is detected by color development using a chemiluminescent substrate such as ECL kit (manufactured by Amersham Bioscience).

When a glycolipid from a cell is used as a sample, the detection of glycolipid may be carried out by extraction from a cell using chloroform, methanol, water or a mixed solvent therewith, separating the resulting glycolipid with thin layer chromatography, and thereafter detecting directly on thin layer chromatograph. Alternatively, the detection of glycolipid can be carried out by transfer of the glycolipid onto PVDF membrane from a thin layer chromatograph after development using an iron or TLC thermal blotter (trade name; manufactured by ATTO), followed by the detection of the glycolipid on the membrane after transfer as in the case of the above-mentioned glycoprotein, with a lectin.

In the separation method of the present invention, a means for separating the pluripotent hepatic progenitor cell with lectin in a step (b) includes, but not particularly limited to, for example, a general cell separation method may be used. The cell separation method includes, for example, a cell fractionation method with flow cytometry, a cell fractionation method with magnetic beads, and a separation method with lectin column chromatography using a resin with lectin bound thereto. The details of these methods are described, for example, in Current Electrophoresis Experimental Method, edited by Japanese Electrophoresis Society published by Ishiyaku Publishers, Inc., New Cell Technology Experimental Protocol, Cell Technology supplementary volume 8 published by Syujunsha Co. Ltd., and the like.

Lectin flow cytometry can be carried out, for example, as follows: A cell used as a material, for example, a cell obtained from the liver (hepatocyte) is washed twice with PBS. PBS containing 0.02 wt % EDTA is added to the cell after washing, and allowed to stand for approximately 5 minutes. Thereafter, the resulting mixture is suspended well. The resulting suspension is centrifuged at 2000 rpm, to remove the supernatant. The resulting cell is washed with PBS, and suspended in PBS to obtain a cell suspension. About 5 μg/ml of labeled lectin is mixed with the cell suspension, and thereafter the resulting mixture is allowed to stand at an appropriate temperature for an appropriate time. Since the specificity of lectin for a sugar chain structure is known to vary depending on a temperature during the incubation, it is preferable to appropriately set a temperature and a time during incubation depending on the lectin used. After the termination of the reaction with lectin, the cell is washed three times with PBS. The sample obtained by suspending the cell in PBS is subjected to Fluorescence activated cell sortor (FACS), to fractionate a cell population specifically labeled with lectin and having strong fluorescent intensity. By the above-mentioned procedures, a cell population containing a viable hepatic progenitor cell can be obtained.

When flow cytometry is carried out, a labeling substance for labeled lectin can be appropriately selected depending on, for example, kinds of laser light and kinds of a filter of a flowcytometer. Specifically, the labeling substance includes, for instance, fluorescein isothiocyanate (FITC), phycoerythrin (PE), allophycocyanin (APC), Texas Red (TR) and the like.

In the identification method and the separation method of a pluripotent hepatic progenitor cell of the present invention, as described above, an antibody capable of binding to the sugar chain expressed (existing) on the pluripotent hepatic progenitor cell (antibody against the sugar chain), for example, an anti-sugar chain antibody capable of specifically binding to a sugar chain structure recognized by the lectin, or a derivative of the antibody can be also used. Namely, in another embodiment of the identification method of the present invention, a sugar chain expressed on a pluripotent hepatic progenitor cell is detected by using the antibody capable of binding to the sugar chain. In such embodiment of the identification method, specifically, a process comprising:

(A′) contacting the “material for separating the pluripotent hepatic progenitor cell” with an antibody capable of binding to the sugar chain expressed on the pluripotent hepatic progenitor cell, and

(B′) detecting a cell with the antibody bound thereto, in the mixture obtained in the step (A′);

is performed to identify the cell with the antibody bound thereto as a pluripotent hepatic progenitor cell.

In addition, in another embodiment of the separation method of the present invention, the pluripotent hepatic progenitor cell is sorted by using the antibody capable of binding to the sugar chain expressed on the pluripotent hepatic progenitor cell. In such an embodiment of the separation method, specifically, a process comprising:

(a′) contacting the “material for separating the pluripotent hepatic progenitor cell” with an antibody capable of binding to the sugar chain expressed on the pluripotent hepatic progenitor cell, and

(b′) sorting to separate a cell with the antibody bound thereto in the mixture obtained in the step (a′);

is carryied out to sort the cell with the antibody bound thereto as a pluripotent hepatic progenitor cell.

In another aspect, the “antibody capable of specifically binding to the sugar chain antigen (antibody against the sugar chain)” used in the identification method and the separation method of the present invention means an antibody capable of specifically binding to the above-mentioned sugar chain expressed (or existing) on the pluripotent hepatic progenitor cell, but not substantially binding to the sugar chain expressed (or existing) on the other cells and not expressed (or existing) on the pluripotent hepatic progenitor cell.

The above-mentioned antibody against the sugar chain can be obtained, for example, by immunizing an animal directly using a sugar chain having an oligosaccharide structure recognized by the lectin used in the present invention. In addition, when the sugar chain has low antigenicity, it is desirable that an animal is immunized using preferably a product obtained by conjugating the sugar chain to a carrier protein or the like, or an animal is immunized directly using a cell expressing a desired sugar chain. As the carrier protein, a protein such as albumin, and KLH (keyhole limpet hemagglutinin), MHC class II peptide and the like are used. In addtion, as the carrier protein, a generally commercially available maleimide activated protein may be used. Alternatively, a protein or a peptide as a carrier, and a sugar chain, a glycopeptide, a glycolipid or a lysoglycolipid as an antigen may be used by conjugating them using an appropriate commercially available linker. In addition, the specificity of the antibody or a derivative thereof used in the present invention can be examined by evaluating as an index of specificity for a sugar chain expressed (or existing) on the pluripotent hepatic progenitor cell, that the antibody reacts with a sugar chain expressed (or existing) on the pluripotent hepatic progenitor cell to show a sedimentation line, and does not react with a control substance different from the sugar chain (e.g. another sugar chain) to show no sedimentation line, by Ouchterlony method using a sugar chain expressed (or existing) on the pluripotent hepatic progenitor cell, and a control substance different from the sugar chain (e.g. another sugar chain); analyzing by kinetic analysis with a surface plasmon resonance method using a substance such as the antibody or a derivative thereof or a sugar chain immobilized onto a matrix and a solution system containing a substance corresponding to the substance immobilized onto the matrix, and the like.

An antibody capable of specifically binding to a sugar chain antigen (antibody against the sugar chain) used in the identification method and the separation method of the present invention is not particularly limited as long as the antibody has ability of specifically binding to the sugar chain antigen, and may be a polyclonal antibody or a monoclonal antibody. Further, in the identification method and the separation method of the present invention, there can be used an antibody modified by the known technique or a derivative of an antibody, for example, a humanized antibody, Fab fragment, F(ab′)₂ fragment, a single chain antibody and the like.

The above-mentioned antibody can be easily prepared, for example, by the method described in Current Protocols in Immunology edited by John E. Coligan published by John Wiley & Sons, Inc. in 1992. Alternatively, the antibody can be prepared by genetic engineering. The antibody can be purified by the conventional method. Further, the resulting antibody is purified, and then treated with a peptidase or the like, to obtain a fragment of the antibody. Specifically, the Fab fragment can be obtained by digesting the antibody against the sugar chain, particularly, a monoclonal antibody with papain. In addition, the F(ab′)₂ fragment can be obtained by digesting the antibody against a sugar chain, particularly, a monoclonal antibody with pepsin. The resulting purified antibody or a derivative thereof can be labeled with enzyme or fluorescence, to use for the identification method or the separation method of the present invention.

In the identification method of the present invention, when a pluripotent hepatic progenitor cell is identified by an anti-sugar chain antibody (antibody against a sugar chain), there can be used, for example, Western blotting method, antibody column method, cell staining method using a labeled antibody, flow cytometry using a labeled antibody, and the like, but not particularly limited thereto.

In the separation method of the present invention, when a pluripotent hepatic progenitor cell is separated by the anti-sugar chain antibody (antibody against a sugar chain), a general cell separation method may be used, but not particularly limited thereto. The above-mentioned cell separation method includes a separation method with magnetic beads using an antibody, a separation method with affinity chromatography using a resin with an antibody bound thereto, a separation method with an antibody matrix with an antibody immobilized thereon, a separation method with a flowcytometer and the like.

In the identification method of the present invention, when a pluripotent hepatic progenitor cell is identified, the expression of a gene for a glycosyltransferase which synthesizes a sugar chain specifically expressed (existing) on the hepatic progenitor cell, the expression of the glycosyltransferase protein or a level of enzyme activity may be measured. Namely, in still another embodiment of the identification method of the present invention, a sugar chain is detected via an expression of an enzyme involved in synthesis of the sugar chain expressed on the pluripotent hepatic progenitor cell. In such embodiment, an expression of an enzyme is detected by means such as the measurement of enzyme activity, the measurement of an amount of an enzyme protein, the measurement of an amount of a mRNA from a gene encoding the enzyme, and the like.

The glycosyltransferase includes N-acetylglucosaminyltransferase III, sialyltransferase, α-1,6 fucosyltransferase and the like. The measurement of an expression level of these glycosyltransferase genes, an expression level of an enzyme protein, and a level of an enzyme activity can be carried out by, for example, referencing to the methods described in The Journal of Biochemistry, vol. 113, p. 692-698 (1993) regarding N-acetylglucosaminyltransferase III, in The Journal of Biochemistry, vol. 120, p. 1-13 (1996) regarding sialyltransferase, in The Journal of Biochemistry, vol. 121, p. 626-632 (1997) regarding α-1,6 fucosyltransferase, or references cited in these references. The expression level of the gene can be measured by Northern blotting method or PCR method by preparing an appropriate probe and/or primer pair based on a nucleotide sequence of a gene encoding each enzyme. The primer used in the present invention can be prepared by, for example, the conventional software assisting design of primers, considering Tm value, the known sequences, the formation of a secondary structure in a nucleic acid and the like. In addition, the probe used in the present invention can be prepared by, for example, the conventional software, considering Tm value, the known sequences, the formation of a secondary structure in a nucleic acid and the like, as in the above-mentioned primer. Specifically, a probe or primer pair may be selected so that it satisfies the optional condition:

(1) There is no significant sequence identity, when a nucleotide sequence of nucleic acid encoding a glycosyltransferase to be measured and the known sequence registered in database are appropriately aligned by BLAST algorithm [condition of Cost to open gap: 5, Cost to extend gap: 2, Penalty for nucleotide mismatch: −3, expect value: 10 and word size: 11] available in the home page address http.//www.ncbi.nlm.nih.gov/BLAST/, to calculate,

(2) The sequence is a sequence by which a secondary structure is hardly formed,

(3) In the case of the primer pair, a strand length is preferably 50 to 8 nucleotides, more preferably 30 to 12 nucleotides and, in the case of the probe, the chain length is preferably 500 to 8 nucleotides, more preferably 100 to 12 nucleotides.

The expression level of the enzyme protein can be measured by Western blotting method using an antibody recognizing each enzyme. In addition, the enzyme activity can be measured using an appropriate acceptor sugar chain and a donor sugar nucleotide, according to the method described, for example, in Glycobiology Experimental Protocol, Cell Technology supplementary volume, Experimental Protocol Series published by Shujunsya in 1996.

Another aspect of the present invention is a method for producing a composition containing a pluripotent hepatic progenitor cell, comprising separating a pluripotent hepatic progenitor cell by the separation method of the present invention. According to the production method of the present invention, since the separation method of the present invention is used, a composition comprising pluripotent hepatic progenitor cell having high purity can be produced. In addition, according to the production method of the present invention, since a substantially homogeneous composition comprising a pluripotent hepatic progenitor cell can be produced, there can be provided a composition comprising a pluripotent hepatic progenitor cell, suitable for application to any of the tissue regeneration by cell transplantation, ex vivo introduction of a nucleic acid molecule, a gene, for example, a therapeutic gene (e.g. gene associated with metabolism in the liver, gene associated with maintenance of homeostasis in the liver, gene encoding protein which is synthesized in the liver (e.g. plasma protein and the like), and the like), a derivative thereof or the like, the production of a transplantation tissue, laboratory use, and the like.

Ingredients other than a pluripotent hepatic progenitor cell contained in the composition comprising a pluripotent hepatic progenitor cell includes, but not particularly limited to, the known cell maintaining ingredients such as saline, cell culturing medium, plasma, serum; extracellular matrix such as collagen, fibronectin, laminin, proteoglycan, glucosaminoglycan and the like. Particularly preferably, there can be used ingredients by which a hepatic progenitor cell can be proliferated or maintained in the state where pluripotency or ability to differentiate into a hepatocyte is maintained.

The production method of the present invention includes a method of carrying out steps (a) and (b) in the above-mentioned separation method, and a method of carrying out steps (a′) and (b′) in the above-mentioned separation method.

A purity of the pluripotent hepatic progenitor cell obtained by the present method can be confirmed using as an index the presence or absence of the expression of CD49f, CD219, c-kit, CD34, Thy-1, albumin, or CK19 in addition to the above-mentioned sugar chain. In addition, the pluripotent hepatic progenitor cell obtained by the present method can be confirmed to be a hepatic progenitor cell, for example, by examining an ability to differentiate into a bile ductal cell or a hepatocyte.

In addition, the pluripotent hepatic progenitor cell obtained by the present method can be evaluated using as an index the detection of an increase in the expression of CK19 gene, a marker of a bile ductal cell and an increase in the expression of albumin gene, a marker of a hepatocyte cell as well as the cell morphology, by maintaining the cell under conditions suitable for inducing the differentiation into a bile ductal cell or a hepatocyte, for example, conditions for incubation at 37° C. in 5 wt % CO₂ in the presence of 1 μM 5-azacytidine or 1 μM retinoic acid and 10 wt % fetal bovine serum, for the differentiation into a bile ductal cell, or conditions for incubation at 37° C. in 5 wt % CO₂ in the presence of a growth factor such as 5 ng/ml HGF (hepatocyte growth factor), 10 ng/ml EGF or 40 ng/ml FGF (fibroblast growth factor), and 10 wt % fetal bovine serum, for the differentiation into a hepatocyte.

As a use of the pluripotent hepatic progenitor cell identified or separated by the identification method or the separation method of the present invention, as well as the composition comprising pluripotent hepatic progenitor cell obtained by the present production method, a hepatic progenitor cell can be administered to the liver surgically or using a catheter and, alternatively, may be administered intravascularly, in regenerative medicine for the liver which was excised due to the treatment of hepatitis, hepatic cancer, and the like. Also in gene therapy, it is expected that a therapeutic gene can be expressed in the liver for a long period of time by introducing a therapeutic gene into the pluripotent hepatic progenitor cell identified or separated by the identification method or the separation method of the present invention, and thereafter administering the resulting hepatic progenitor cell to the liver or intravascularly in the same manner.

Still another aspect of the present invention is a method for targeting a pluripotent hepatic progenitor cell, comprising contacting a substance to be introduced with the pluripotent hepatic progenitor cell and/or introducing the substance to be introduced into a pluripotent hepatic progenitor cell, wherein the sugar chain expressed on the pluripotent hepatic progenitor cell is used as a target.

The method for targeting a pluripotent hepatic progenitor cell of the present invention enables delivery of a drug or a gene to a hepatic progenitor cell by utilizing specific binding or specific association between the “sugar chain expressed on the pluripotent hepatic progenitor cell” and the “factor capable of specifically binding to, or associating with the sugar chain expressed on the pluripotent hepatic progenitor cell” in the conventional technique of the drug delivery or the gene delivery.

In the method for targeting a pluripotent hepatic progenitor cell of the present invention, when the drug delivery is carried out, there may be used a drug delivery product obtained by allowing a carrier suitable for the drug delivery to carry the “factor capable of specifically binding to, or associating with a sugar chain expressed on a pluripotent hepatic progenitor cell”, or a drug delivery product obtained by allowing the drug itself to carry the “factor capable of specifically binding to, or associating with a sugar chain expressed on a pluripotent hepatic progenitor cell” in a range that pharmacological property of the drug is exhibited. In addition, when the gene delivery is carried out in the method for targeting a pluripotent hepatic progenitor cell of the present invention, there may be used a gene delivery product obtained by allowing a gene-transferring carrier (liposome and the like) carrying a gene to be introduced, to carry the “factor capable of specifically binding to, or associating with a sugar chain expressed on the pluripotent hepatic progenitor cell”.

Further, in the method for targeting a pluripotent hepatic progenitor cell of the present invention, when the gene delivery is performed, a retroviral vector carrying a gene to be introduced may be used in the presence of a mixture comprising at least a functional substance A (polypeptide and the like) comprising a retrovirus-binding site (e.g. retrovirus binding site such as fibroblast growth factor, collagen, polylysine and the like) and a functional substance B (polypeptide and the like) comprising a site involved in binding or association of the “factor capable of specifically binding to, or associating with the sugar chain expressed on the pluripotent hepatic progenitor cell” with a sugar chain, or an artificial substrate comprising the functional substance A and the functional substance B. The mixture or the artificial substrate may further comprise another cell adhesion factor. Each of the contents of the functional substance A and the functional substance B in the mixture may be an amount suitable for binding or association between retrovirus and the functional substance A and binding or association between a pluripotent hepatic progenitor cell and the functional substance B.

The “factor which specifically binds to or associates with a sugar chain expressed on a pluripotent hepatic progenitor cell” includes an antibody recognizing the lectin or the sugar chain.

The method for targeting a pluripotent hepatic progenitor cell of the present invention can be evaluated by examining an ability of binding to a sugar chain by surface plasmon resonance analysis using, for example, a sugar chain immobilized onto a matrix and a solution comprising a drug delivery product, a solution comprising a gene delivery product or a solution comprising a combination of a retroviral vector and the mixture or the artificial substrate.

The method for targeting a pluripotent hepatic progenitor cell of the present invention is advantageous in the case where a nucleic acid molecule, a gene, for example, a therapeutic gene (e.g. gene associated with metabolism in the liver, gene associated with maintenance of homeostasis in the liver, gene encoding protein which is synthesized in the liver, and the like), a derivative thereof or the like is introduced ex vivo for disease in the liver, disease accompanied with decrease in function in the liver and the like.

The present invention will be explained in detail below by Examples, but the present invention is not limited by the Examples.

### EXAMPLE 1

As a pluripotent hepatic progenitor cell strain, a rat liver epithelial (RLE) cell at around 20 passage provided by Dr. Thorgeirsson of NIH was used. As a control cell, human hepatoblastoma cell Huh6 (JCRB0401, JCRB cell bank), human hepatoblastoma cell Huh7 (JCRB0403, JCRB cell bank), rat hepatic cancer cell m31 (JCRB0422), mouse melanoma cell B16F10 (ATCC CRL-6475), mouse fetal hepatocyte BLN-CL (ATCC TIB-73), and mouse fetal hepatic cancer cell BNL-A7 were used.

All of the above-mentioned cells were cultured by incubation at 37° C. in 5% by volume CO₂ in a medium containing 10 wt % fetal bovine serum (FCS) and 50 μg/ml of kanamycin. As the medium, Ham-F12 medium (manufactured by ICN biomedicals, Inc) for the RLE cell, RPMI-1640 medium (manufactured by SIGMA) for the Huh6 cell and the Huh7 cell, Dulbecco's modified Eagle's medium (D-MEM) for the m31 cell, the B16F10 cell, the BNL-CL cell and the BNL-A7 cell were used.

Cultured cells were collected from a culture petri dish, and thereafter the resulting cultured cells were washed once with phosphate buffered saline (PBS). Thereafter, to the resulting cell, TNE buffer [10 mM Tris-HCl, pH 7.8 containing 1 wt % NP-40 (Nonidet P-40), 0.15 M NaCl, as well as 1 mM EDTA and protease inhibitor (10 μM aprotinin) was added, thereby solubilizing the cell.

To an amount corresponding to 20 μg of a protein of the solubilized cell lysate, SDS-PAGE sample buffer [composition: 10 mM Tris-HCl, pH 6.8, 2 wt % SDS, 1 wt % 2-mercaptoethanol, 8M urea was added and thereafter the resulting mixture was incubated at 100° C. for 5 minutes, to denature the protein. The resulting sample was subjected to electrophoresis (SDS-PAGE) using 10 wt % SDS-polyacrylamide gel. After termination of electrophoresis, the separated protein on the gel was blotted onto a nitrocellulose membrane. The nitrocellulose membrane was blocked in 3 wt % bovine serum albumin (BSA) at room temperature overnight.

Next, using lectin, the protein on the nitrocellulose membrane was analyzed as follows: As the lectin, biotinylated lectin [manufactured by HONEN CORPORATION] was used. Lectin used is kidney bean (Phaseolus vulgaris) lectin E4PHA and wheat germ lectin WGA.

Lectin was diluted 1000-fold with Tris buffered saline (TBS) containing 0.05 wt % Tween 20 (trade name). The nitrocellulose membrane after blocking was immersed in the resulting solution, and incubated at room temperature for 30 minutes. Thereafter, the nitrocellulose membrane was washed three times with TBS containing 0.05 wt % Tween 20 (trade name) for 10 minutes.

In addition, peroxidase-labeled avidin [trade name: a reagent contained in ABC kit (manufactured by Vector Laboratories) was diluted 2000-fold with TBS containing 0.05 wt % Tween 20 (trade name), and thereafter the nitrocellulose membrane after washing was immersed in the resulting solution and incubated at room temperature for 30 minutes. Thereafter, the membrane after incubation was washed three times with TBS containing 0.05 wt % Tween 20 for 10 minutes. The membrane after washing was developed using ECL kit (trade name; manufactured by Amersham Biosciences).

As a result, a protein from a pluripotent hepatic progenitor cell strain, RLE cell, was very strongly stained with E4PHA as compared with a protein from the hepatoblastoma cell, the hepatic cancer cell or the fetal hepatocyte. Therefore, it is suggested that a sugar chain recognized by E4PHA is predominantly or specifically expressed on a pluripotent hepatic progenitor cell strain.

### EXAMPLE 2

Each of the pluripotent hepatic progenitor cell strain RLE cell used in Example 1 and, as control cells, a rat ascites hepatic cancer cell AH66, a hepatocyte immediately after perfusion, a hepatocyte and a hepatic non-parenchymal cell after culturing for 48 hours was analyzed with a flowcytometer using various lectins. The hepatocyte immediately after perfusion is a hepatocyte obtained from 9 to 12 week-old SD rat, by the conventional in situ perfusion method, and the hepatocyte and the hepatic non-parenchymal cell after culturing for 48 hours are a cell obtained by obtaining a hepatocyte and a hepatic non-parenchymal cell from 9 to 12 week-old SD-rat, by the in situ perfusion method, and culturing the resulting hepatocyte and hepatic non-parenchymal cell on a collagen-coated dish under the conditions of 5% by volume CO₂ and 37° C., using D-MEM comprising 10 wt % FCS, 100 mg/l kanamycin (manufactured by Sigma), 0.1 μM insulin and 10 μM dexamethasone (manufactured by Wako Pure Chemical Industries, Ltd.), while the medium was exchanged with fresh medium every 3 to 6 hours.

All of the above-mentioned cells were cultured by incubation on a collagen-coated dish at 37° C. in 5 wt % CO₂ using a medium containing 10 wt % FCS and 50 μg/ml kanamycin. As the medium, Ham-F12 medium (manufactured by ICN biomedicals, Inc) for the RLE cell, D-MEM medium containing 0.1 μM insulin and 10 μM dexamethasone for the cultured hepatocyte were used.

The cultured cells were collected from a culture dish, and washed twice with 10 milliliters of PBS. To the resulting cell, PBS containing 1 ml of 0.02 wt % EDTA was added, and thereafter the resulting suspension was allowed to stand at room temperature for 5 minutes. Thereafter, the resulting cells were suspended well, and the resulting suspension was transferred to an 1.5 ml Eppendorf tube. A 5 μg/ml FITC-labeled lectin was added to 100 μl of PBS, and thereafter the resulting solution and the cell suspension in the Eppendorf tube were mixed. The resulting mixture was incubated at room temperature for 15 minutes. Thereafter, the cell was washed three times with 1 ml of PBS and, finally, suspended in 0.5 ml of PBS. The resulting cell suspension was subjected to FACS. The cell with lectin bound thereto was quantitated by using a difference between fluorescent intensity at 480 nm resulting from FITC lectin and fluorescent intensity of spontaneous emission of a sample without any staining, namely, the cell suspension sample without FITC-labeled lectin. As a lectin, LCA and E4PHA were used. In addition, as a control, Canavalia ensiformis lectin [Concanavalin A (ConA)] was used.

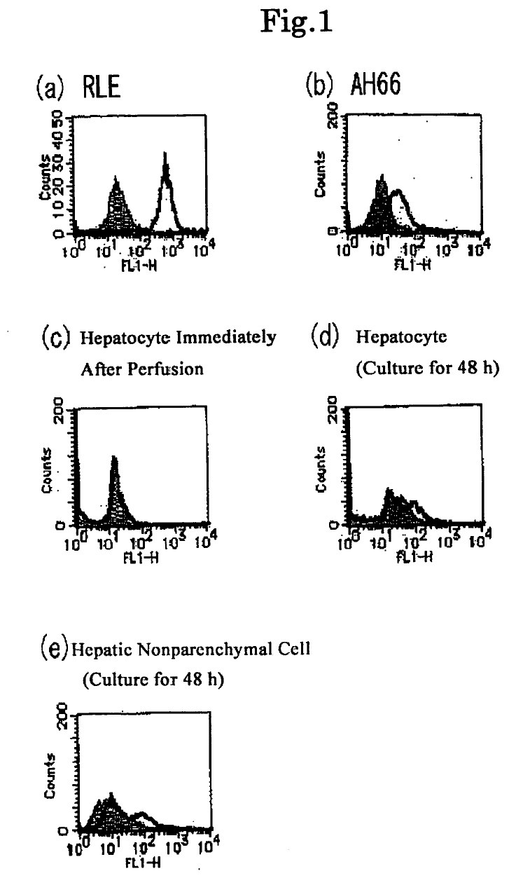

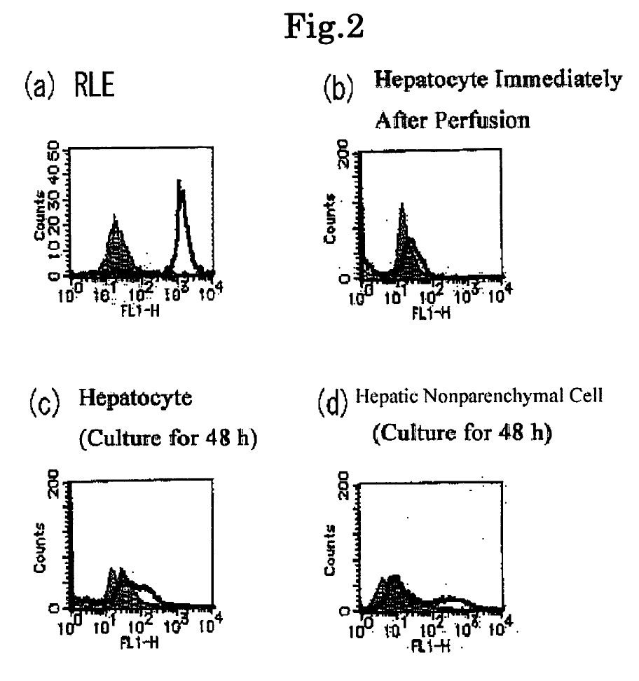

The results of FACS using FITC-labeled E4PHA are shown in FIG. 1. In addition, the results of FACS using FITC-labeled LCA are shown in FIG. 2.

As shown in FIG. 1, a pluripotent hepatic progenitor cell, RLE cell, was more strongly stained with FITC-labeled E4PHA as compared with control cells, a hepatic cancer cell AH66, a perfused hepatocyte and a hepatic non-parenchymal cell. In addition, as shown in FIG. 2, the RLE cell was also more strongly stained with FITC-labeled LCA as compared with a hepatocyte and a hepatic non-parenchymal cell.

Further, an experiment was carried out using WGA as described above. As a result, it was revealed that the RLE cell was also stained with WGA. In addition, a hepatic non-parenchymal cell and a hepatic cancer cell had also affinity with WGA.

### EXAMPLE 3

A cell in the living state was separated by identifying a difference in a sugar chain on the cell surface by binding with lectin.

RLE cell was cultured at 37° C. in 5% by volume CO₂ for 24 hours in a 10 cm dish containing Ham-F12 medium containing 10 wt % fetal bovine serum (FCS) and 50 μg/ml kanamycin in the presence of 10 ng/ml EGF (epidermal growth factor) and 1 mM nicotinamide. Sub-confluent cells on the dish were treated with trypsin and then collected (10⁷ or more cells). Collected cells were washed with PBS, and suspended in 5 ml of PBS. Biotinylated E4PHA was added to the resulting suspension so as to give a concentration of 10 μg/ml, followed by rotation at 4° C. for 5 minutes.

Next, cells were washed with PBS, and then suspended in 80 μl of PBS. To the resulting cell suspension, 20 μl of trade name: Streptavidin Microbeads (manufactured by Daiichi Pure Chemicals Co., Ltd.) was added and mixed. The resulting mixture was incubated at 4° C. for 15 minutes. Thereafter, the resulting cells were washed with PBS, and suspended in 500 μl of PBS. Using the resulting cell suspension and trade name: autoMACS (manufactured by Daiichi Pure Chemical Co., Ltd.), a cell bound to E4PHA (RLE cell) was separated from a cell not bound to E4PHA.

The cell bound to E4PHA was tested as shown in Example 5 below, and was confirmed to have ability to differentiate into a bile ductal cell or a hepatocyte.

### EXAMPLE 4

RLE cell and as a control cells, m31 cell and a hepatocyte immediately after perfusion were investigated for the presence of GnT-III (N-acetylglucosaminyltransferase-III) which is a glycosyltransferase involved in the production of an oligosaccharide structure bisected GlcNAc recognized by E4PHA lectin.

Each cell was solubilized, and subjected to SDS-PAGE and blotting to a nitrocellulose membrane in the same manner as that of Example 1. Thereafter, GnT-III was detected using a mouse-derived anti-GnT-III antibody (see The Journal of Biochemistry, vol. 278, p. 25295-25301) as a primary antibody, and HRP (horseradish peroxidase)-labeled anti-mouse antibody (manufactured by Promega) as a secondary antibody.

As a result, GnT-III was detected in the RLE cell, but the presence of GnT-III was not found in the m31 or the hepatocyte.

From the above results, it was revealed that a hepatic progenitor cell could be detected using the presence of GnT-III as an index.

### EXAMPLE 5

Ability of differentiation of RLE cell was investigated by inducing the RLE cell to differentiate into a bile ductal cell or a hepatocyte.

A 32-passage RLE cell was cultured at 37° C. in 5 wt % CO₂ for 10 days in Ham-F12 medium (manufactured by ICN biomedicals, Inc.) containing 10 wt % fetal bovine serum (FCS) and 50 μg/ml kanamycin in the presence of 1 μM 5-azacytidine (manufactured by SIGMA) or 1 μM all-trans retinoic acid (manufactured by SIGMA). As a control, a 32-passage RLE cell was cultured similarly in the absence of 1 μM 5-azacytidine (manufactured by SIGMA) or 1 μM all-trans retinoic acid (manufactured by SIGMA).

Separately, a 24-passage RLE cell was cultured at 37° C. in 5 wt % CO₂ for 7 days in a Ham-F12 medium (manufactured by ICN biomedicals, Inc) containing 10 wt % fetal bovine serum (FCS) and 50 μg/ml kanamycin in the presence of a growth factor [5 ng/ml HGF (hepatocyte growth factor), 10 ng/ml EGF or 40 ng/ml FGF (fibroblast growth factor)]. As a control, a 24-passage RLE cell was cultured similarly in the absence of the above-mentioned growth factor.

A total RNA was extracted from the cell after culturing by the conventional method. Using the resulting total RNA, RT-PCR was carried out as follows: Specifically, a 1 st strand cDNA was synthesized using the above-mentioned total RNA, trade name: Reverse Transcription System (manufactured by Promega) and TaKaRa Ex Taq (TAKARA BIO INC). Using the resulting cDNA as a template, PCR was performed to investigate the expression of each of albumin gene, cytokeratin 19 (CK19) gene and, as a control, β-actin gene. As a primer pair in the PCR, the following primer pair:

a primer pair of a sense primer (GAGAAGGTCACCAAGTGCTGTAGT; SEQ ID NO: 1) and an antisense primer (CTGGGAGTGT GCAGATATCA GAGT; SEQ ID NO: 2) for albumin gene,

a primer pair of a sense primer (ACCATGCAGAACCTGAACGAT; SEQ ID NO: 3) and an antisense primer (CACCTCCAGCTCGCCATTAG; SEQ ID NO: 4) for CK19 gene, or

a primer pair of a sense primer (GAAGATTTGGCACCACACTTT; SEQ ID NO: 5) and an antisense primer (TTGAATGTAGTTTCATGGAT; SEQ ID NO: 6) for β-actin gene.

By these primer pairs, a 141 bp DNA fragment for albumin gene, a 83 bp DNA fragment for cytokeratin 19 (CK19) gene, and a 595 bp DNA fragment for β-actin gene are obtained. PCR thermal profiles are 40 cycles, one cycle being denaturation: 95° C. for 30 seconds, annealing: 70° C. for 1 minute and extension: 72° C. for 1 minute and 30 seconds for albumin gene, 32 cycles, one cycle being denaturation: 95° C. for 30 seconds, annealing: 62° C. for 1 minute and extension: 72° C. for 1 minute and 30 seconds for CK19 gene, and 30 cycles, one cycle being denaturation: 95° C. for 30 seconds, annealing: 55° C. for 1 minute and extension: 72° C. for 1 minute and 30 seconds for β-actin gene. The resulting amplification products were confirmed by electrophoresis on 2 wt % agarose gel.

From the results of RT-PCR, it was revealed that the expression of a marker of a bile ductal cell, CK19 gene, was highly increased in the RLE cell cultured in the presence of retinoic acid. In addition, the expression of albumin gene was slightly increased in the RLE cell cultured in the presence of retinoic acid. On the other hand, a slight increase in the expression of CK19 gene was seen also in the RLE cell cultured in the presence of 5-azacytidine.

In addition, an increase in the expression of a marker of a hepatocyte cell, albumin gene, was found in the RLE cell cultured in the presence of EGF, and in the RLE cell cultured in the presence of various growth factors. To the contrary, an increase in the expression of albumin gene was not found in the cell cultured in the presence of HGF or FGF. In addition, the expression of CK19 gene was not seen in any of cells.

From the above results, it was revealed that the RLE cell was a precursor cell capable of differentiating into bile ductal cell or a hepatocyte.

Sequence Listing Free Text

SEQ ID NO: 1 is a sequence of sense primer for albumin gene.

SEQ ID NO: 2 is a sequence of antisense primer for albumin gene.

SEQ ID NO: 3 is a sequence of sense primer for CK19 gene.

SEQ ID NO: 4 is a sequence of antisense primer for CK19 gene.

SEQ ID NO: 6 is a sequence of sense primer for β-actin gene.

SEQ ID NO: 7 is a sequence of antisense primer for β-actin gene.

Equivalent

The invention may be embodied in other specific forms without departing from the spirit or essential characteristics thereof. Examples mentioned in the above are embodiment is therefore to be considered in all respects as illustrative and not restrictive, the scope of the invention being indicated by the appended claims rather than by the foregoing description and all changes which come within the meaning and range of equivalency of the claims are therefore intended to be embraced therein.

What is claimed is:

1. A method for separating a pluripotent hepatic progenitor cell, comprising sorting the pluripotent hepatic progenitor cell using as an index a sugar chain expressed on the pluripotent hepatic progenitor cell.

2. The method according to claim 1, wherein the pluripotent hepatic progenitor cell is sorted by using a protein capable of binding to the sugar chain expressed on the pluripotent hepatic progenitor cell.

3. The method according to claim 2, wherein the protein is a lectin capable of binding to the sugar chain expressed on the pluripotent hepatic progenitor cell.

4. The method according to claim 2, wherein the sugar chain expressed on the pluripotent hepatic progenitor cell comprises a sugar chain structure recognized by at least one lectin selected from the group consisting of kidney bean lectin, wheat germ lectin, lentil lectin and Aleuria aurantia lectin.

5. The method according to claim 1, wherein the pluripotent hepatic progenitor cell is sorted by using an antibody capable of binding to the sugar chain expressed on the pluripotent hepatic progenitor cell.

6. A method for producing a composition comprising a pluripotent hepatic progenitor cell, comprising the step of separating the pluripotent hepatic progenitor cell by the method of any one of claims 1 to 5.
